(19) United States
(12) Patent Application Publication
(10) Pub. No.: US 2018/0333505 A1
(43) Pub. Date: Nov. 22, 2018

(54) SELECTIVE INSERTION OF SUICIDE GENES INTO CANCER CELLS

(71) Applicants: Matthew J. Lin, East Lyme, CT (US); Ryan Englander, Suffield, CT (US)

(72) Inventors: Matthew J. Lin, East Lyme, CT (US); Ryan Englander, Suffield, CT (US)

(21) Appl. No.: 15/601,102

(22) Filed: May 22, 2017

(51) Int. Cl. A61K 48/00; A61P 35/00; A61P 35/04; A61K 31/522; C12N 9/22; C12N 15/11; C12N 15/85; A61K 38/45

(52) U.S. Cl. CPC A61K 48/005; A61P 35/00; A61P 35/04; A61K 31/522; C12N 9/22; C12N 2800/80; C12N 15/85; A61K 38/45; C12Y 207/01021; C12N 2310/20; C12N 15/11

(57) ABSTRACT

A method for treating cancer by inducing a double strand DNA break at a chromosomal site in cancer cells, inserting a nucleic acid encoding a suicide gene into the chromosomal site having the double strand DNA break, and expressing the suicide gene in the cancer cells, thereby inducing cell death upon expression of the suicide gene. The chromosomal site includes a protospacer adjacent motif (PAM) absent from a corresponding chromosomal site in normal cells. Also disclosed is a cancer treatment method that includes identifying a single nucleotide change in the genomic sequence of cancer cells from a subject as compared to the genomic sequence of normal cells, the single nucleotide change forming a PAM, inserting a suicide gene specifically into the cancer cell genomic sequence, and activating the suicide gene, thereby leading to cell death. Furthermore, provided is a clustered regularly interspaced short palindromic repeat-associated protein having RNA-guided DNA endonuclease activity.

### SEQUENCE LISTING

A computer readable file containing a sequence listing is being electronically co-filed herewith via EFS-Web. The computer readable file, submitted under 37 CFR § 1.821(e), will also serve as the copy required by 37 § CFR 1.821(c). The file (filename “2AP6744.TXT”) was created on May 12, 2017 and has a size of 50,872 bytes.

The content of the computer readable file is hereby incorporated by reference in its entirety.

### BACKGROUND

Clustered Regularly Interspaced Short Palindromic Repeat-associated protein (CRISPR/Cas) systems have revolutionized genome editing, allowing for rapid disruption of genes or insertion of foreign genetic material at targeted loci by inducing site-specific double-stranded breaks (DSBs). The DSBs can then be repaired by error-prone non-homologous end joining (NHEJ) or high-fidelity homology-directed repair (HDR). Most CRISPR/Cas systems accomplish this via a guide RNA (gRNA) complementary to a sequence adjacent to a protospacer adjacent motif (PAM) specific to the Cas enzyme being used. In the absence of a PAM sequence, the Cas enzyme will not create the DSB.

Manipulating the portion of the gRNA that hybridizes to the targeted loci allows for the induction of a DSB anywhere in the genome that contains an adjacent PAM. Donor DNA of interest may be inserted at the site of this DSB via HDR, provided that the donor DNA is present in the cell at the time the DSB is made. The donor DNA must also contain information localizing it to the DSB, which is accomplished through the use of homology arms on either side of the donor DNA that is to be inserted.

While the specificity of CRISPR/Cas systems is high, it is not sufficiently high to target cell types based on characteristic single nucleotide changes, as single base pair mismatches between the gRNA and target DNA are tolerated. Further, the use of Cas enzymes in a clinical context has been inhibited by its relatively high degree of off-target activity.

In the context of cancer treatment, the use of “suicide genes” that kill cells expressing them upon the application of a nontoxic prodrug have seen widespread use but have been limited by the specificity of their respective delivery systems. For example, the thymidine kinase from herpes simplex virus phosphorylates the nontoxic nucleoside analogue ganciclovir, after which the product may be further phosphorylated and incorporated into DNA, ultimately killing the cell.

Methods to introduce suicide gene systems specifically into malignant cells have the potential to concomitantly vastly increase the efficiency of suicide gene-mediated tumor killing while dramatically reducing off-target cytotoxicity.

The need exists to develop methods for treating cancer that better spare normal cells and tissues without losing effectiveness against cancer cells.

### SUMMARY

To meet the need discussed above, a first method for treating cancer is provided that includes the steps of (i) inducing a double strand DNA break at a chromosomal site in cancer cells of a subject suffering from cancer, (ii) inserting a nucleic acid encoding a suicide gene into the chromosomal site having the double strand DNA break, and (iii) expressing the suicide gene in the cancer cells. Expression of the suicide gene, which is expressed specifically in the cancer cells, leads to cell death.

The chromosomal site includes a PAM absent from the corresponding chromosomal site in normal cells. As such, DSBs are induced specifically in the cancer cells and not in the normal cells.

Also provided is a second method for treating cancer. The second method includes obtaining a first genomic sequence from a normal cell of a subject suffering from a cancer; obtaining a cancer tissue sample from the subject, obtaining a second genomic sequence from the cancer tissue sample, identifying a single nucleotide change in the second genomic sequence as compared to the first genomic sequence, the single nucleotide change forming a PAM, designing a gRNA that includes a sequence complementary to a portion of the second genomic sequence immediately adjacent to the PAM, expressing the gRNA in cells of the subject together with an RNA-guided endonuclease that specifically recognizes the PAM, the RNA-guided endonuclease inducing a double strand DNA break in the second genomic sequence and not in the first genomic sequence, introducing into the cells of the subject a vector that contains a nucleic acid encoding a suicide gene, whereby the suicide gene is inserted into the second genomic sequence and not the first genomic sequence, and activating the suicide gene. Upon activation of the suicide gene, the cancer cells undergo cell death.

Furthermore, a CRISPR-associated protein having the amino acid sequence of SEQ ID NO: 1 is disclosed. The CRISPR-associated protein has RNA-guided DNA endonuclease activity. It can be used to carry out the two above-described cancer treatment methods.

The details of one or more embodiments of the invention are set forth in the description below. Other features, objects, and advantages of the invention will be apparent from the description and from the claims.

### DETAILED DESCRIPTION

As mentioned above, the first method for treating cancer requires that a PAM sequence be present in cancer cells as compared to normal cells. The PAM sequence can differ by a single nucleotide as compared to the corresponding chromosomal site in normal cells.

Alternatively, the PAM sequence in cancer cells can differ by two or three nucleotides from the corresponding site in normal cells.

The presence of the PAM sequence allows the induction of a double strand DNA break in a sequence adjacent to the PAM. The double strand DNA break is induced by a clustered regularly interspaced short palindromic repeat-associated protein (Cas). For example, the Cas protein can be Streptococcus pyogenes Cas9 (SpCas9), enhanced SpCas9 (eSpCas9), SpCas9-high fidelity 1 (SpCas9-HF1), Prevotella and Francisella clustered regularly interspaced short palindromic repeat-associated protein 1 (Cpf1), and an RNA-guided endonuclease having the amino acid sequence of SEQ ID NO: 1 (eSpCas9-HF1).

Any rationally designed improvements on the above-mentioned Cas proteins can be used in the cancer treatment methods described herein. In one example, the Cas protein is a catalytically inactive variant (“dead” Cas9 or dCas9) fused to a nondiscriminatory nuclease domain, e.g., a FokI nuclease domain. In another example, the Cas protein is a nickase variant in which one of the catalytic domains has been inactivated (SpCas9n), fused to a nondiscriminatory nuclease domain, e.g., a FokI nuclease domain.

As set forth, supra, the methods disclosed herein result in the insertion of a suicide gene specifically in cancer cells. In this context, the term “specifically” means that the suicide gene is inserted into the cancer cells at least 100 to 10000-fold (e.g., 100, 200, 300, 400, 500, 600, 700, 800, 900, 1000, 2000, 3000, 4000, 5000, 6000,7000, 8000, 9000, and 10000-fold) more efficiently as compared to normal cells.

The suicide gene can be, but is not limited to, a viral thymidine kinase, thymidine kinase from herpes simplex virus (HSV-TK), splice-corrected HSV-TK (scHSV-TK), TK.007, a cytosine deaminase, a caspase, a DNase, an RNase, a bacterial nitroreductase (e.g., E. coli nitroreductase), a uracil phosphoribosyltransferase, a linamarase (e.g., E. coli linamarase), a carboxyl esterase, an intracellular antibody against vital enzymes, an intracellular aptamer against vital enzymes, a purine nucleoside phosphorylase, carboxypeptidase G2, E. coli β-galactosidase, and hepatic cytochrome P450-2B1.

After insertion of the suicide gene into the cancer cells, the suicide gene is expressed, thereby resulting in cell death of the cancer cell. The inserted suicide gene can be operably linked to a constitutively expressed promoter. Alternatively, the suicide gene is operably linked to a cancer-specific promoter.

Moreover, the suicide gene can be operably linked to an inducible expression system. For example, the inducible expression system can be a tetracycline-regulated system, e.g., a Tet-On system and a Tet-Off system. In these systems, tetracycline can be introduced to activate or suppress expression of the suicide gene.

In a particular aspect of the method, the suicide gene kills cancer cells by activating its latent activity, for example by enhancing the anti-cancer activity of a chemotherapy drug or by converting a prodrug into an active anti-cancer drug. In a particular example, viral thymidine kinase converts ganciclovir into a toxic product in cells expressing thymidine kinase. In another example, the non-toxic nucleoside analogue 5-fluorocytosine is converted by cytosine deaminase into toxic 5-fluorouracil. Other non-limiting examples include converting 5-flurouracil into active intermediate 5-fluorouridine-5′-monophosphate with uracil phosphoribosyltransferase, converting metronidazole with E. coli nitroreductase into an active metabolite, activating fludarabine by E. coli purine nucleoside phosphorylase, activating doxorubicin by carboxypeptidase G2, converting linamarin to cyanide by linamarase, activating N-[4″-(beta-D-galactopyranosyl)-3″-nitrobenzyl oxycarbonyl] daunomycin (Daun02) upon cleavage by E. coli β-galactosidase, and potentiating the activity of cyclophosphamide by expressing hepatic cytochrome P450-2B1.

The above method can also include inducing double strand breaks at more than one chromosomal site specifically in cancer cells. For example, cancer cell genomic sequences can differ from that of normal cells at more than one distinct chromosomal site, each difference giving rise to a PAM. A double strand DNA break can be induced in a sequence adjacent to each PAM. In an exemplary method, 1-20 (e.g., 1, 2, 3, 4, 5, 6, 7, 8, 9, 10, 11, 12, 13, 14, 15, 16, 17, 18, 19 and 20) double strand DNA breaks are induced in each cancer cell, each double strand break at a distinct chromosomal site. The suicide genes set forth above can be inserted into each distinct chromosomal site. A single suicide gene can be inserted into each chromosomal site or more than one distinct suicide gene can be inserted, each into a distinct chromosomal site.

The method set forth above can be used for treating cancer. Examples of cancers that can be treated include bladder cancer (e.g., transitional cell carcinoma, squamous cell carcinoma, adenocarcinoma, small-cell carcinoma, and sarcoma) brain cancer (e.g., astrocytoma, glioblastoma multiforme, meningioma, ependymoma, oligodendroglioma, mixed glioma, macroadenoma, microadenoma, craniopharyngioma, germinoma, pineoblastoma, pineocytoma, and medulloblastoma), breast cancer (e.g., ductal carcinoma in situ, invasive ductal carcinoma, lobular carcinoma, inflammatory breast cancer, male breast cancer, metastatic breast cancer, Paget's disease of the breast, and papillary carcinoma), cervical cancer (e.g., squamous cell carcinoma and adenocarcinoma), colorectal cancer (e.g., colorectal adenocarcinoma, gastrointestinal carcinoid tumors, gastrointestinal stromal tumors, and leiomyosarcoma), esophageal cancer (e.g., adenocarcinoma and squamous cell carcinoma), kidney cancer (e.g., clear cell renal cell carcinoma, papillary renal cell carcinoma, chromophobe renal cell carcinoma, collecting duct renal cell carcinoma, unclassified renal cell carcinoma, transitional cell carcinoma, and renal sarcoma), liver cancer (e.g., hepatocellular carcinoma, fibrolamellar hepatocellular carcinoma, cholangiocarcinoma, and angiosarcoma), lung cancer (e.g., small cell carcinoma, combined small cell carcinoma, squamous cell non-small cell lung cancer, adenocarcinoma, and large-cell undifferentiated carcinoma), melanoma (e.g., cutaneous melanomas, ocular melanoma, and mucosal melanoma), ovarian cancer (e.g., epithelial ovarian cancer, germ cell ovarian cancer, and sex cord-stromal ovarian cancer), pancreatic cancer (e.g., adenocarcinoma and endocrine tumors), prostate cancer (e.g., adenocarcinoma and sarcoma), skin cancer (basal cell carcinoma, squamous cell carcinoma, Kaposi's sarcoma, Merkel cell carcinoma, skin adnexal tumors, and sarcomas), thyroid cancer (e.g., papillary carcinoma, follicular carcinoma, Hiirthle cell carcinoma, medullary thyroid carcinoma, and anaplastic carcinoma), uterine cancer (e.g., endometrial cancer and uterine sarcoma), metastatic cancer, leukemia, and lymphoma.

The second method for treating the above-listed cancers is carried out by obtaining a first genomic sequence from a normal cell of a subject suffering from a cancer, obtaining a cancer tissue sample from the subject, obtaining a second genomic sequence from the cancer tissue sample, and identifying a single nucleotide change in the second genomic sequence as compared to the first genomic sequence, the single nucleotide change forming a PAM in cancer cells in the cancer tissue sample.

The PAM recognized by SpCas9, eSpCas9, and SpCas9-HF1 is NGG, thus any single nucleotide change in the sequences NN_{A/C/T}G and NGN_{A/C/T} to NGG in cancer cells creates a PAM targetable by those enzymes. The PAM for Cpf1 is TTN; hence, a single nucleotide change in the sequences TN_{A/C/G}N and N_{A/C/G}TN to TTN is targetable by Cpf1.

The second method for treating cancer includes designing a guide RNA (gRNA), having a sequence complementary to a portion of the second genomic sequence immediately adjacent to the PAM. More specifically, the sequence that the PAM is adjacent to would be incorporated as the portion of the gRNA in the Cas RNP complex that determines sequence specificity. For example, the PAM for SpCas9, eSpCas9, and SpCas9-HF1 is immediately 3′ of the target sequence. The approximately 17-23 nucleotide sequence immediately 5′ of the PAM would be targeted for insertion of the suicide gene. In another example, the PAM for Cpf1 is immediately 5′ of the target sequence. As such, the approximately 17-23 nucleotide sequence immediately 3′ of the knocked-in PAM would be targeted for insertion of the suicide gene.

The gRNA thus designed is then expressed in cells of the subject together with an RNA-guided endonuclease that specifically recognizes the PAM. The RNA-guided endonuclease can be, but is not limited to SpCas9, eSpCas9, SpCas9-HF1, Cpf1, or an RNA-guided endonuclease having the amino acid sequence of SEQ ID NO: 1 (eSpCas9-HF1). Additional RNA-guided endonucleases are described, supra.

In a particular embodiment of the second method, expressing the gRNA is accomplished by synthesizing the gRNA in vitro, incubating it with the RNA-guided endonuclease to form a complex, and introducing the complex into the cells of the subject.

Upon introducing the complex into the cells, the RNA-guided endonuclease induces a double strand DNA break in the second genomic sequence, i.e., in the cancer cells, and not in the first genomic sequence of normal cells.

After introducing double strand breaks specifically in the cancer cells, a vector that contains a nucleic acid encoding a suicide gene is introduced into the cells of the subject.

In order to insert the suicide gene at the site of the double strand break, the vector includes two homology arms flanking the suicide gene. The homology arms each contain regions of extensive homology to the DNA surrounding the desired insertion site, i.e., the site of the double strand break. For example, each of the two homology arms can be 500 bp to 1000 bp in length. The degree of homology between each homology arm and the DNA surrounding the insertion site is at least 99%.

The Cas enzyme and vector must both be present in the cancer cell concurrently to achieve selective insertion of the suicide gene. To accomplish this, the Cas enzyme and gRNA can be encoded on a single plasmid alongside the DNA to be inserted. The plasmid can be delivered to cells via many methods, including but not limited to a viral vector (e.g., adenovirus, lentivirus, retrovirus, virus-like particles), lipofection, electroporation, nanoparticles, cell-penetrating peptides, cationic polymers, calcium phosphate, and magnetofection.

Alternatively, the genes encoding the Cas enzyme and gRNA may be carried on a plasmid separate from the vector containing the suicide gene and homology arms.

The vector can be in the form of a plasmid, linear double-stranded DNA, or linear single-stranded DNA. In a particular example, the vector is a viral vector. In another specific example, the viral vector is non-tissue-specific. In one embodiment, the viral vector is tissue-specific.

In another example, the vector may be introduced into cells together with purified Cas already complexed with gRNA (“Cas RNP”). Furthermore, a viral gag-Cas RNP fusion protein can be generated, which allows the packaging of Cas RNP fusion protein into virus-like particles and subsequent intracellular delivery into target cells.

The above steps of the second method result in insertion of the suicide gene specifically into the cancer cells and not into normal cells.

The suicide genes discussed above with respect to the first cancer treatment method can also be used in the second method.

After insertion of the suicide gene, it is activated, thereby causing the cancer cell to undergo cell death. Again, activation of the suicide gene is accomplished as set forth above for the first method.

The second method, like the first method can also include inducing double strand breaks at more than one chromosomal site specifically in cancer cells. In an example, a plurality of single nucleotide changes are identified in the cancer tissue genomic sequence as compared to the first genomic sequence, each single nucleotide change forming a PAM in cancer cells in the cancer tissue sample. A gRNA can be designed to target a sequence adjacent to each PAM. For example, double strand DNA breaks in the cancer cells can be induced adjacent to 1-20 (e.g., 1, 2, 3, 4, 5, 6, 7, 8, 9, 10, 11, 12, 13, 14, 15, 16, 17, 18, 19 and 20) PAM. The suicide gene can be inserted at the site of each double strand break using multiple vectors, each of which includes two homology arms containing regions of extensive homology to the DNA surrounding each of the desired insertion sites. Each vector can also include a distinct suicide gene.

In an embodiment of the second cancer treatment method, genes encoding RNAi constructs that abrogate the expression of key genes involved in the NHEJ pathway are delivered into cells together with the suicide gene vector and the Cas RNP. Doing so increases the efficiency of the HDR pathway, leading to increased insertion of the suicide gene. Additionally, the method can include administering compounds that upregulate the HDR pathway. RS-1 and L755507 are two examples of such compounds.

In some methods, in addition to or instead of inserting a suicide gene, a Cas enzyme is targeted to the sequence immediately adjacent to a cancer cell specific PAM and the resultant DSB is repaired by NHEJ, producing a gene knockout. In an example, the knocked-out gene is an oncogene driving a malignant phenotype. In another example, the knocked-out gene is a mutated gene of interest suspected to be driving a mutant phenotype. Further, the knocked-out gene can be a mutated gene that transforms a genetic element/cassette from a cooperative to a selfish genetic element. Additionally, the knocked-out gene can be a mutated gene conferring resistance to a particular treatment.

In some embodiments, agents, chemical, biological, or otherwise, that modulate the frequency with which different mechanisms are employed to repair Cas-induced DSBs may be delivered alongside the other components of the aforementioned methods.

Also disclosed is the use of an RNA-guided endonuclease for inserting a suicide gene specifically into a cancer cell, where the RNA-guided endonuclease induces a double strand DNA break at a chromosomal site in the cancer cell and not at a corresponding chromosomal site in normal cells. The chromosomal site in the cancer cell contains a single nucleotide difference forming a PAM that is absent from normal cells.

Further disclosed is the use of a composition comprising an RNA-guided endonuclease, a gRNA, and a vector that includes a suicide gene flanked by two homology arms, for killing cancer cells, in which the RNA-guided endonuclease induces a double strand DNA break at a chromosomal site containing a PAM specifically in a cancer cell and not at a corresponding chromosomal site in normal cells and the suicide gene is inserted at the site of the double strand DNA break.

The specific example below is to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### Example: PAM Sequences Identified in a Cancer Cell Line

The genome of FABF cells, a cancer cell line derived from C57BL/6 mice, was sequenced and compared to the genome sequence of wild-type C57BL/6 animals. Single nucleotide differences in the cancer cell genome giving rise to a PAM sequence were identified. The results are shown in Table 1 below.

TABLE 1
PAM Sequences in Cancer Cell Line

| | SEQ | | Chro- | |
|---|---|---|---|---|
| Cancer | ID | Wild- | mo- | |
| Cell PAM Sequence | NO. | type | some | Location |
| ttttaagatttatttattttaGg | 3 | aAg | 1 | 34985753 |
| gaagagggcatcagatccattgG | 4 | tgA | 1 | 34985807 |
| caaatcccagcaaccacatggGg | 5 | gTg | 1 | 34985823 |
| tcaaatcccagcaaccacatggG | 6 | ggT | 1 | 34985823 |
| actgactgctctaaggccctgGg | 7 | gAg | 1 | 34985848 |
| cactgactgctctaaggccctgG | 8 | tgA | 1 | 34985848 |
| tatctccatcttcagggccacGg | 9 | cCg | 1 | 58420163 |
| aacggcatagactttgaaagtgG | 10 | tgC | 1 | 84963968 |
| gaaagtatgcattgttcacaggG | 11 | ggT | 1 | 88265846 |
| agatcttgtccagatacaactGg | 12 | tAg | 1 | 88270078 |
| cttatttggcccagtacagagGg | 13 | gAg | 1 | 88272689 |
| acttatttggcccagtacagagG | 14 | agA | 1 | 88272689 |
| cctgaggccaatcagcaaactGg | 15 | tAg | 1 | 88274942 |
| atagagggacagtttcaggcaGg | 16 | aAg | 1 | 88277238 |
| acttcatttttggtatagaagGg | 17 | gAg | 1 | 112457577 |
| aacttcatttttggtatagaagG | 18 | agA | 1 | 112457577 |
| ctccttgacaagattccatttgG | 19 | tgT | 1 | 118543321 |
| cattaaaacaatgactagatgGg | 20 | gAg | 1 | 136273753 |
| tcattaaaacaatgactagatgG | 21 | tgA | 1 | 136273753 |
| taagttatgacaaaggagaatgG | 22 | tgC | 1 | 160940917 |
| ttactatcaggaatgccttttGg | 23 | tCg | 10 | 26276115 |
| tgtcatagggaagggggagtgGg | 24 | gTg | 10 | 56497362 |
| ttgtcatagggaagggggagtgG | 25 | tgT | 10 | 56497362 |
| cttagatttgaccttagtacaGg | 26 | aAg | 10 | 75937468 |
| taagcctagggctagaaaatagG | 27 | agT | 10 | 80644313 |
| aaatgttttactctgctgagcGg | 28 | cAg | 11 | 3150561 |
| aaactaaagacatctgggaagGg | 29 | gTg | 11 | 3678257 |
| caaactaaagacatctgggaagG | 30 | agT | 11 | 3678257 |
| cagagtgtcattaatagtgttGg | 31 | tAg | 11 | 4215137 |
| accagcaagattttgctgaagGg | 32 | gTg | 11 | 35783452 |
| taccagcaagattttgctgaagG | 33 | agT | 11 | 35783452 |
| tggtttacgaggttcctatggGg | 34 | gAg | 11 | 58040587 |
| gtggtttacgaggttcctatggG | 35 | ggA | 11 | 58040587 |
| ttgaaattcttaacaagaagcGg | 36 | cTg | 11 | 74660426 |
| accacaccttgtgtccattccGg | 37 | cCg | 11 | 76247440 |
| agagatgcctgggaaagctctGg | 38 | tTg | 11 | 77563671 |
| tgataaagcaatctttcactgGg | 39 | gAg | 11 | 102308940 |
| gtgataaagcaatctttcactgG | 40 | tgA | 11 | 102308940 |
| cctaggcaggtggccctagggGg | 41 | gTg | 11 | 109011727 |
| ccctaggcaggtggccctagggG | 42 | ggT | 11 | 109011727 |
| ccctaggcaggtggccctaggGg | 43 | gCg | 11 | 109011728 |
| tccctaggcaggtggccctaggG | 44 | ggC | 11 | 109011728 |
| cacagtgggcttggcccttctgG | 45 | tgA | 11 | 109011783 |
| attgtgttaattattcattgaGg | 46 | aAg | 11 | 109011784 |

| | | | | |
|---|---|---|---|---|
| taattaacacaatctcctgtggG | 47 | ggA | 11 | 109011815 |
| aaaagttaattgggatgatatGg | 48 | tAg | 11 | 109011936 |
| gcagccaaaagttaattgggaGg | 49 | aAg | 11 | 109011942 |
| acatggcacctgcaatcagctgG | 50 | tgA | 11 | 109012035 |
| cctgtgtccctaccagacccaGg | 51 | aCg | 12 | 73112753 |
| acagaatggtgagaatgtaaggG | 52 | ggA | 12 | 73123829 |
| ctattatttttgttagaggtggG | 53 | ggC | 12 | 77448660 |
| gtgtaccttccctgaaagaggGg | 54 | gAg | 12 | 78350929 |
| tgtgtaccttccctgaaagaggG | 55 | ggA | 12 | 78350929 |
| attttgagatagtcatagcctGg | 56 | tCg | 12 | 85926817 |
| tcagcaagcagttttattagaGg | 57 | aCg | 12 | 102474650 |
| gcactctggaaacatacaacaGg | 58 | aTg | 13 | 45551487 |
| gagtggagtggaaggggtttgGg | 59 | gTg | 13 | 66007874 |
| tgagtggagtggaaggggtttgG | 60 | tgT | 13 | 66007874 |
| gaattcctttccccagacccggG | 61 | ggA | 13 | 72630777 |
| atataaagaactcaagaaggtGg | 62 | tCg | 13 | 97163121 |
| aggtaacagggtagattagtcGg | 63 | cCg | 14 | 14737424 |
| ctctgcatgctgaaggcttctgG | 64 | tgT | 14 | 19418754 |
| aagctaaaacacttctgagttgG | 65 | tgA | 14 | 103130018 |
| gatttccagagtggttgtataGg | 66 | aAg | 15 | 8351189 |
| tacctagtgtgccggaaggtagG | 67 | agA | 15 | 75086819 |
| ggaccagtcgctctcagacaggA | 68 | ggA | 15 | 75086861 |
| tagaagcgcaatgtgccaaaaGg | 69 | aTg | 15 | 80155817 |
| cggatgggctgaatgggtcacGg | 70 | cAg | 15 | 81649577 |
| gagttggatctctcctattgtgG | 71 | tgC | 16 | 14120302 |
| gtttgaccctataaccttcacGg | 72 | cAg | 16 | 17222325 |
| tgtagtagcctattttctcttgG | 73 | tgA | 16 | 91534663 |
| tggcacaactggttgttatcgGg | 74 | gTg | 17 | 6295728 |
| ctggcacaactggttgttatcgG | 75 | cgT | 17 | 6295728 |
| gtagccaacatcaaagtaaatgG | 76 | tgA | 17 | 15739614 |
| ctgcaagggctatttctaaaaGg | 77 | aCg | 17 | 27116715 |
| gatgagcagagtttcttatatgG | 78 | tgC | 17 | 32056768 |
| cagaggagaccaagagaacatGg | 79 | tCg | 17 | 32368078 |
| cacttcaagacaaatggattagG | 80 | agA | 17 | 36231338 |
| acactggcatcagaggatacagG | 81 | agT | 17 | 36231489 |
| aatacccacctctgcaaaaggGg | 82 | gAg | 17 | 39843435 |
| caatacccacctctgcaaaaggG | 83 | ggA | 17 | 39843435 |
| ctgctttctgatgggacgtaaGg | 84 | aAg | 17 | 39843842 |
| atacatgtataacttgtgcatgG | 85 | tgC | 17 | 39843971 |
| gcacccacatagcaggtcatagG | 86 | agT | 17 | 39844034 |
| tcatttatgtattatgcatgtgG | 87 | tgC | 17 | 39844684 |
| gggcgaggctgagtggttaaaGg | 88 | aAg | 17 | 39844879 |
| gtgtcctagcaaaaagggaggGg | 89 | gAg | 17 | 39845049 |
| tgtgtcctagcaaaaagggaggG | 90 | ggA | 17 | 39845049 |
| ctctgtgctgaaggcagcagtGg | 91 | tTg | 17 | 39845219 |
| ggagaagatggcaaaactactgG | 92 | tgT | 17 | 39845224 |
| caagaggcatcgagattgcatgG | 93 | tgA | 17 | 39846189 |
| ttatcctccttcatcaccaccGg | 94 | cAg | 17 | 39846199 |
| gcggaagagggaggcagcaggGg | 95 | gAg | 17 | 39848233 |
| ggcggaagagggaggcagcaggG | 96 | ggA | 17 | 39848233 |
| tctatgtgatgctgagctgatgG | 97 | tgA | 17 | 47000678 |
| ctgggttctttccagatgctggG | 98 | ggA | 17 | 70963741 |
| catatcagccttatttataatGg | 99 | tTg | 17 | 70963742 |
| ggcctttaaggatcccaaactGg | 100 | tTg | 18 | 3004843 |
| ggaaaaggggaggaggagaggGg | 101 | gCg | 18 | 11084560 |
| gggaaaaggggaggaggagaggG | 102 | ggC | 18 | 11084560 |
| aatgtggaagttctgtccttgGg | 103 | gAg | 18 | 40308151 |
| taatgtggaagttctgtccttgG | 104 | tgA | 18 | 40308151 |
| cagtgaaatttatttctgggagG | 105 | agT | 18 | 75001023 |
| tcacctctggcatcttaaactGg | 106 | tCg | 19 | 5884443 |
| ctaacatggtttgacatcaaaGg | 107 | aTg | 2 | 5379370 |
| tgtttcgagacagggtttctcGg | 108 | cCg | 2 | 22739599 |
| ttcaaggccagcctggtctacGg | 109 | cAg | 2 | 22739630 |
| tcctagcacttgggaggcagagG | 110 | agC | 2 | 22739668 |
| gtaacaaggcagggccggtaggG | 111 | ggA | 2 | 22745088 |
| ctggtaacagggcagggtgttGg | 112 | tCg | 2 | 29065469 |
| taaatcaggaaagaaaaagaagG | 113 | agA | 2 | 41987795 |
| aaggcagaagaccataataatGg | 114 | tAg | 2 | 41987962 |
| atgttatagaatgtttttagtgG | 115 | tgC | 2 | 65099110 |
| aataagtcaccttactgtccagG | 116 | agT | 2 | 75980007 |
| taagaccacccaaactctgccGg | 117 | cCg | 2 | 125114043 |
| aggaaggaaggaagggagggaGg | 118 | aAg | 2 | 149743534 |
| gacccacaaacattctaccctGg | 119 | tAg | 2 | 157901494 |
| cttgggaggcagaggcaggcagG | 120 | agA | 2 | 164429721 |
| tggacttctctgaattaacctGg | 121 | tCg | 2 | 166611642 |
| ggaagagattgtgaatggagggG | 122 | ggA | 2 | 168940784 |
| cctatcttgggaggctgtagaGg | 123 | aAg | 3 | 56828650 |
| atctccttttgttgtcgaattgG | 124 | tgA | 3 | 89224316 |
| caactcaccctcttaactttggG | 125 | ggA | 3 | 95734876 |
| tagcaaaaacaaaacgaaaagGg | 126 | gAg | 3 | 106043261 |
| atagcaaaaacaaaacgaaaagG | 127 | agA | 3 | 106043261 |
| gctcttcctcgtactcttttcgG | 128 | cgT | 3 | 106043269 |
| ttgtttttgctatttccaagagG | 129 | agA | 3 | 106043293 |
| ttctgaataaccaaaactctcGg | 130 | cAg | 3 | 106043403 |
| tacacacccaggtgtcccagggG | 131 | ggC | 3 | 142810824 |
| ggcatttcatctaaggttcttGg | 132 | tCg | 4 | 21873684 |
| atgcatatttgcttatgtaaaGg | 133 | aAg | 4 | 43493458 |
| atgaccaacaaagggtggtaaGg | 134 | aAg | 4 | 45399747 |
| aagaagatacaaatttatagcGg | 135 | cAg | 4 | 88641162 |
| agaagatacaaatttatagcagG | 136 | agC | 4 | 88641164 |
| tttttggtatatattttaggaGg | 137 | aCg | 4 | 88641218 |
| tttttttcctcctgcaaatctgG | 138 | tgT | 4 | 94582584 |
| aggaaccgccctgaggagagcGg | 139 | cTg | 4 | 136994438 |
| ctgccacatgggggcaacagagG | 140 | agC | 4 | 141476596 |
| gaaggatactgacgtccaggcgG | 141 | cgA | 4 | 146263964 |
| tcctgctaaaaaattactaacgG | 142 | cgC | 4 | 147745994 |
| ggctccacaccccgtggtcgagG | 143 | agC | 4 | 147863009 |
| ttctgtttacatgggctgtgtgG | 144 | tgC | 5 | 34634791 |
| attgctggtcaacaatggattGg | 145 | tCg | 5 | 34965744 |
| tagacttgagcaatgacagagGg | 146 | gTg | 5 | 109599044 |
| atagacttgagcaatgacagagG | 147 | agT | 5 | 109599044 |
| gggtcctctggaagaacagcaGg | 148 | aCg | 5 | 120749516 |
| cacgttgatcaatggaattaaGg | 149 | aCg | 5 | 146261004 |
| ccaccatttccaatttcaagtGg | 150 | tAg | 5 | 146261076 |
| gaaacaatcctgaacagtgaaGg | 151 | aAg | 5 | 146261113 |
| gaattttggtgggtatctcacGg | 152 | cTg | 5 | 146261271 |
| cctgtctgtctctgcctctctgG | 153 | tgA | 6 | 3201546 |
| cagagagagacagagacagagGg | 154 | gAg | 6 | 3201552 |
| gcagagagagacagagacagagG | 155 | agA | 6 | 3201552 |
| cacaccacaacccctacacctGg | 156 | tCg | 6 | 21986807 |
| tatgtaaaaatgacacgattaGg | 157 | aCg | 6 | 71129302 |
| gggatgttgtgtaggagcttggG | 158 | ggT | 6 | 108471108 |
| aatgggtctctagttgcgggtGg | 159 | tCg | 6 | 125494593 |
| tggttacagtagccgcagcacGg | 160 | cAg | 6 | 128202046 |
| ccgctttacttctgatagattgG | 161 | tgA | 7 | 18818744 |
| aacctaagacaggtacagtccGg | 162 | ccG | 7 | 35392845 |
| gtctggaagcttttctgtgtggG | 163 | ggC | 7 | 80760902 |
| tattccctgatagatcataagGg | 164 | gAg | 7 | 127136919 |
| gtattccctgatagatcataagG | 165 | agA | 7 | 127136919 |
| agatagaaggagatggtacatGg | 166 | tCg | 7 | 130759981 |
| gatttttctctatagtgcagcGg | 167 | cCg | 7 | 141151710 |
| tatcccttgagcagtaaaggaGg | 168 | aAg | 7 | 141178481 |
| ttttctggtagaattttatgagG | 169 | agC | 8 | 14975927 |
| tttaaaaccctgtttccagcagG | 170 | agC | 8 | 61329366 |
| ttccacaaaatagaaacaaaagG | 171 | agC | 8 | 71255446 |
| gcattaatcccttctaagggcGg | 172 | cCg | 8 | 121556313 |
| ggagttccttgagccactcaagG | 173 | agC | 9 | 3258817 |
| actcaagtctaaaaggatcaagG | 174 | agA | 9 | 3258832 |
| acttatagaggagaaagtggggG | 175 | ggA | 9 | 3258883 |
| ttcagggaacctggtgagggagG | 176 | agA | 9 | 17030907 |
| acaggacaatgtgggtagaagGg | 177 | gAg | 9 | 104215765 |
| aacaggacaatgtgggtagaagG | 178 | agA | 9 | 104215765 |
| ttttagcagaagagcattttggG | 179 | ggA | 9 | 110068727 |
| agagaaatcagatctcagcgagG | 180 | agA | 9 | 110281249 |
| cttaataaccgacatcattatGg | 181 | tTg | 9 | 110281264 |

The single nucleotide difference in each cancer cell genomic sequence is capitalized. A PAM sequence is present in the last three nucleotides of each cancer cell genomic sequence fragment. The wild-type sequence of these three nucleotides is shown in the third column, also with the single nucleotide difference capitalized.

The results indicated that many single nucleotide changes that occur in cancer cells give rise to PAM sequences. As such, many CRISPR/Cas targetable sites can be found in cancer cells that do not exist in the corresponding normal cells.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

1. A method for treating cancer, the method comprising:
inducing a double strand DNA break at a chromosomal site in cancer cells of a subject suffering from cancer,
inserting a nucleic acid encoding a suicide gene into the chromosomal site having the double strand DNA break, and
expressing the suicide gene in the cancer cells, whereby the cancer cells undergo cell death upon expression of the suicide gene,
wherein the chromosomal site includes a protospacer adjacent motif (PAM) absent from a corresponding chromosomal site in normal cells of the subject and the double strand break is induced specifically in the cancer cells and not in the normal cells.

2. The method of claim 1, wherein the PAM differs by a single nucleotide as compared to the corresponding chromosomal site in the normal cells of the subject.

3. The method of claim 2, wherein the double strand DNA break is induced by Streptococcus pyogenes clustered regularly interspaced short palindromic repeat-associated protein 9 (SpCas9), enhanced SpCas9 (eSpCas9), SpCas9-high fidelity 1 (SpCas9-HF1), Prevotella and Francisella clustered regularly interspaced short palindromic repeat-associated protein 1 (Cpf1), or an RNA-guided endonuclease having the amino acid sequence of SEQ ID NO: 1 (eSpCas9-HF1).

4. The method of claim 3, wherein the suicide gene is herpes simplex virus thymidine kinase (HSV-TK), HSV-TK.007, cytosine deaminase, uracil phosphoribosyltransferase, a caspase, a DNase, an RNase, intracellular antibodies against vital enzymes, intracellular aptamers against vital enzymes, E. coli nitroreductase, E. coli purine nucleoside phosphorylase, carboxypeptidase G2, linamarase, E. coli β-galactosidase, or hepatic cytochrome P450-2B1.

5. The method of claim 4, wherein expressing the suicide gene is accomplished by activating a latent activity of the suicide gene.

6. The method of claim 5, wherein the suicide gene is HSV-TK.007 and the latent activity is activated with ganciclovir.

7. The method of claim 6, wherein the double strand DNA break is induced by eSpCas9, SpCas9-HF1, or eSpCas9-HF1.

8. The method of claim 1, wherein the double strand DNA break is induced by SpCas9, eSpCas9, SpCas9-HF1, Cpf1, or eSpCas9-HF1.

9. The method of claim 8, wherein the suicide gene is herpes simplex virus thymidine kinase (HSV-TK), HSV-TK.007, cytosine deaminase, uracil phosphoribosyltransferase, a caspase, a DNase, an RNase, intracellular antibodies against vital enzymes, intracellular aptamers against vital enzymes, E. coli nitroreductase, E. coli purine nucleoside phosphorylase, carboxypeptidase G2, linamarase, E. coli β-galactosidase, or hepatic cytochrome P450-2B1.

10. The method of claim 1, wherein the cancer is bladder cancer, brain cancer, breast cancer, cervical cancer, colorectal cancer, esophageal cancer, kidney cancer, liver cancer, lung cancer, melanoma, ovarian cancer, pancreatic cancer, prostate cancer, skin cancer, thyroid cancer, uterine cancer, or metastatic cancer.

11. A method for treating cancer, the method comprising:
obtaining a first genomic sequence from a normal cell of a subject suffering from a cancer;
obtaining a cancer tissue sample from the subject;
obtaining a second genomic sequence from the cancer tissue sample;
identifying a single nucleotide change in the second genomic sequence as compared to the first genomic sequence, the single nucleotide change forming a protospacer adjacent motif (PAM);
designing a guide RNA (gRNA), the gRNA including a sequence complementary to a portion of the second genomic sequence immediately adjacent to the PAM;
expressing the gRNA in cells of the subject together with an RNA-guided endonuclease that specifically recognizes the PAM, the RNA-guided endonuclease inducing a double strand DNA break in the second genomic sequence and not in the first genomic sequence;
introducing into the cells of the subject a vector that contains a nucleic acid encoding a suicide gene, whereby the suicide gene is inserted into the second genomic sequence and not the first genomic sequence; and
activating the suicide gene,
wherein cancer cells in the subject undergo cell death upon activation of the suicide gene.

12. The method of claim 11, wherein the RNA-guided endonuclease is Streptococcus pyogenes clustered regularly interspaced short palindromic repeat-associated protein 9 (SpCas9), enhanced SpCas9 (eSpCas9), SpCas9-high fidelity 1 (SpCas9-HF1), Prevotella and Francisella clustered regularly interspaced short palindromic repeat-associated protein 1 (Cpf1), or an RNA-guided endonuclease having the amino acid sequence of SEQ ID NO: 1 (eSpCas9-HF1).

13. The method of claim 12, wherein, the suicide gene is herpes simplex virus thymidine kinase (HSV-TK), HSV-TK.007, cytosine deaminase, uracil phosphoribosyltransferase, a caspase, a DNase, an RNase, intracellular antibodies against vital enzymes, intracellular aptamers against vital enzymes, E. coli nitroreductase, E. coli purine nucleoside phosphorylase, carboxypeptidase G2, linamarase, E. coli β-galactosidase, or hepatic cytochrome P450-2B1.

14. The method of claim 13, wherein activating the suicide gene is accomplished by activating a latent activity of the suicide gene.

15. The method of claim 14, wherein the suicide gene is HSV-TK.007 and the latent activity is activated by administering ganciclovir to the subject.

16. The method of claim 15, wherein the double strand DNA break is induced by eSpCas9, SpCas9-HF1, or eSpCas9-HF1.

17. The method of claim 11, wherein the gRNA, the RNA-guided endonuclease, and the suicide gene are introduced into the cells of the subject in a single vector.

18. The method of claim 11, wherein the expressing step is accomplished by synthesizing the gRNA in vitro, incubating it with the RNA-guided endonuclease to form a complex, and introducing the complex into the cells of the subject.

19. The method of claim 11, wherein the cancer is bladder cancer, brain cancer, breast cancer, cervical cancer, colorectal cancer, esophageal cancer, kidney cancer, liver cancer, lung cancer, melanoma, ovarian cancer, pancreatic cancer, prostate cancer, skin cancer, thyroid cancer, uterine cancer, or metastatic cancer.

20. A clustered regularly interspaced short palindromic repeat-associated protein having the amino acid sequence of SEQ ID NO: 1, wherein the protein has RNA-guided DNA endonuclease activity.


SEQUENCE LISTING (181 sequences)

SEQ ID NO: 1 - protein, 1368 aa, Artificial Sequence
  eSpCas9-HF1 fusion protein
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - DNA, 4104 bp, Artificial Sequence
  Codon-optimized eSpCas9-HF1
atggacaagaagtacagcatcggcctggacatcggcaccaacagcgtgggctgggccgtgatcaccgacgagtacaaggtgcccagcaagaagttcaaggtgctgggcaacaccgacagacacagcatcaagaagaacctgatcggcgccctgctgttcgacagcggcgagaccgccgaggccaccagactgaagagaaccgccagaagaagatacaccagaagaaagaacagaatctgctacctgcaggagatcttcagcaacgagatggccaaggtggacgacagcttcttccacagactggaggagagcttcctggtggaggaggacaagaagcacgagagacaccccatcttcggcaacatcgtggacgaggtggcctaccacgagaagtaccccaccatctaccacctgagaaagaagctggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgccagactgagcaagagcagaagactggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagagtgaacaccgagatcaccaaggcccccctgagcgccagcatgatcaagagatacgacgagcaccaccaggacctgaccctgctgaaggccctggtgagacagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaacagagaggacctgctgagaaagcagagaaccttcgacaacggcagcatcccccaccagatccacctgggcgagctgcacgccatcctgagaagacaggaggacttctaccccttcctgaaggacaacagagagaagatcgagaagatcctgaccttcagaatcccctactacgtgggccccctggccagaggcaacagcagattcgcctggatgaccagaaagagcgaggagaccatcaccccctggaacttcgaggaggtggtggacaagggcgccagcgcccagagcttcatcgagagaatgaccgccttcgacaagaacctgcccaacgagaaggtgctgcccaagcacagcctgctgtacgagtacttcaccgtgtacaacgagctgaccaaggtgaagtacgtgaccgagggcatgagaaagcccgccttcctgagcggcgagcagaagaaggccatcgtggacctgctgttcaagaccaacagaaaggtgaccgtgaagcagctgaaggaggactacttcaagaagatcgagtgcttcgacagcgtggagatcagcggcgtggaggacagattcaacgccagcctgggcacctaccacgacctgctgaagatcatcaaggacaaggacttcctggacaacgaggagaacgaggacatcctggaggacatcgtgctgaccctgaccctgttcgaggacagagagatgatcgaggagagactgaagacctacgcccacctgttcgacgacaaggtgatgaagcagctgaagagaagaagatacaccggctggggcgccctgagcagaaagctgatcaacggcatcagagacaagcagagcggcaagaccatcctggacttcctgaagagcgacggcttcgccaacagaaacttcatggccctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggcagacacaagcccgagaacatcgtgatcgagatggccagagagaaccagaccacccagaagggccagaagaacagcagagagagaatgaagagaatcgaggagggcatcaaggagctgggcagccagatcctgaaggagcaccccgtggagaacacccagctgcagaacgagaagctgtacctgtactacctgcagaacggcagagacatgtacgtggaccaggagctggacatcaacagactgagcgactacgacgtggaccacatcgtgccccagagcttcctggccgacgacagcatcgacaacaaggtgctgaccagaagcgacaagaacagaggcaagagcgacaacgtgcccagcgaggaggtggtgaagaagatgaagaactactggagacagctgctgaacgccaagctgatcacccagagaaagttcgacaacctgaccaaggccgagagaggcggcctgagcgagctggacaaggccggcttcatcaagagacagctggtggagaccagagccatcaccaagcacgtggcccagatcctggacagcagaatgaacaccaagtacgacgagaacgacaagctgatcagagaggtgaaggtgatcaccctgaagagcaagctggtgagcgacttcagaaaggacttccagttctacaaggtgagagagatcaacaactaccaccacgcccacgacgcctacctgaacgccgtggtgggcaccgccctgatcaagaagtaccccgccctggagagcgagttcgtgtacggcgactacaaggtgtacgacgtgagaaagatgatcgccaagagcgagcaggagatcggcaaggccaccgccaagtacttcttctacagcaacatcatgaacttcttcaagaccgagatcaccctggccaacggcgagatcagaaaggcccccctgatcgagaccaacggcgagaccggcgagatcgtgtgggacaagggcagagacttcgccaccgtgagaaaggtgctgagcatgccccaggtgaacatcgtgaagaagaccgaggtgcagaccggcggcttcagcaaggagagcatcctgcccaagagaaacagcgacaagctgatcgccagaaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagagaagcagcttcgagaagaaccccatcgacttcctggaggccaagggctacaaggaggtgaagaaggacctgatcatcaagctgcccaagtacagcctgttcgagctggagaacggcagaaagagaatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagagagtgatcctggccgacgccaacctggacaaggtgctgagcgcctacaacaagcacagagacaagcccatcagagagcaggccgagaacatcatccacctgttcaccctgaccaacctgggcgcccccgccgccttcaagtacttcgacaccaccatcgacagaaagagatacaccagcaccaaggaggtgctggacgccaccctgatccaccagagcatcaccggcctgtacgagaccagaatcgacctgagccagctgggcggcgac

SEQ ID NO: 3 - DNA, 23 bp, Artificial sequence
  PAM Knock-in
ttttaagatttatttattttagg

SEQ ID NO: 4 - DNA, 23 bp, Artificial Sequence
  PAM Knock-in
gaagagggcatcagatccattgg

SEQ ID NO: 5 - DNA, 23 bp, Artificial sequence
  PAM Knock-in
caaatcccagcaaccacatgggg

SEQ ID NO: 6 - DNA, 23 bp, Artificial sequence
  PAM Knock-in
tcaaatcccagcaaccacatggg

SEQ ID NO: 7 - DNA, 23 bp, Artificial sequence
  PAM Knock-in
actgactgctctaaggccctggg

SEQ ID NO: 8 - DNA, 23 bp, Artificial sequence
  PAM Knock-in
cactgactgctctaaggccctgg

SEQ ID NO: 9 - DNA, 23 bp, Artificial sequence
  PAM Knock-in
tatctccatcttcagggccacgg

SEQ ID NO: 10 - DNA, 23 bp, Artificial sequence
  PAM Knock-in
aacggcatagactttgaaagtgg

SEQ ID NO: 11 - DNA, 23 bp, Artificial sequence
  PAM Knock-in
gaaagtatgcattgttcacaggg

SEQ ID NO: 12 - DNA, 23 bp, Artificial sequence
  PAM Knock-in
agatcttgtccagatacaactgg

SEQ ID NO: 13 - DNA, 23 bp, Artificial sequence
  PAM Knock-in
cttatttggcccagtacagaggg

SEQ ID NO: 14 - DNA, 23 bp, Artificial sequence
  PAM Knock-in
acttatttggcccagtacagagg

SEQ ID NO: 15 - DNA, 23 bp, Artificial sequence
  PAM Knock-in
cctgaggccaatcagcaaactgg

SEQ ID NO: 16 - DNA, 23 bp, Artificial sequence
  PAM Knock-in
atagagggacagtttcaggcagg

SEQ ID NO: 17 - DNA, 23 bp, Artificial sequence
  PAM Knock-in
acttcatttttggtatagaaggg

SEQ ID NO: 18 - DNA, 23 bp, Artificial sequence
  PAM Knock-in
aacttcatttttggtatagaagg

SEQ ID NO: 19 - DNA, 23 bp, Artificial sequence
  PAM Knock-in
ctccttgacaagattccatttgg

SEQ ID NO: 20 - DNA, 23 bp, Artificial sequence
  PAM Knock-in
cattaaaacaatgactagatggg

SEQ ID NO: 21 - DNA, 23 bp, Artificial sequence
  PAM Knock-in
tcattaaaacaatgactagatgg

SEQ ID NO: 22 - DNA, 23 bp, Artificial sequence
  PAM Knock-in
taagttatgacaaaggagaatgg

SEQ ID NO: 23 - DNA, 23 bp, Artificial sequence
  PAM Knock-in
ttactatcaggaatgccttttgg

SEQ ID NO: 24 - DNA, 23 bp, Artificial sequence
  PAM Knock-in
tgtcatagggaagggggagtggg

SEQ ID NO: 25 - DNA, 23 bp, Artificial sequence
  PAM Knock-in
ttgtcatagggaagggggagtgg

SEQ ID NO: 26 - DNA, 23 bp, Artificial sequence
  PAM Knock-in
cttagatttgaccttagtacagg

SEQ ID NO: 27 - DNA, 23 bp, Artificial sequence
  PAM Knock-in
taagcctagggctagaaaatagg

SEQ ID NO: 28 - DNA, 23 bp, Artificial sequence
  PAM Knock-in
aaatgttttactctgctgagcgg

SEQ ID NO: 29 - DNA, 23 bp, Artificial sequence
  PAM Knock-in
aaactaaagacatctgggaaggg

SEQ ID NO: 30 - DNA, 23 bp, Artificial sequence
  PAM Knock-in
caaactaaagacatctgggaagg

SEQ ID NO: 31 - DNA, 23 bp, Artificial sequence
  PAM Knock-in
cagagtgtcattaatagtgttgg

SEQ ID NO: 32 - DNA, 23 bp, Artificial sequence
  PAM Knock-in
accagcaagattttgctgaaggg

SEQ ID NO: 33 - DNA, 23 bp, Artificial sequence
  PAM Knock-in
taccagcaagattttgctgaagg

SEQ ID NO: 34 - DNA, 23 bp, Artificial sequence
  PAM Knock-in
tggtttacgaggttcctatgggg

SEQ ID NO: 35 - DNA, 23 bp, Artificial sequence
  PAM Knock-in
gtggtttacgaggttcctatggg

SEQ ID NO: 36 - DNA, 23 bp, Artificial sequence
  PAM Knock-in
ttgaaattcttaacaagaagcgg

SEQ ID NO: 37 - DNA, 23 bp, Artificial sequence
  PAM Knock-in
accacaccttgtgtccattccgg

SEQ ID NO: 38 - DNA, 23 bp, Artificial sequence
  PAM Knock-in
agagatgcctgggaaagctctgg

SEQ ID NO: 39 - DNA, 23 bp, Artificial sequence
  PAM Knock-in
tgataaagcaatctttcactggg

SEQ ID NO: 40 - DNA, 23 bp, Artificial sequence
  PAM Knock-in
gtgataaagcaatctttcactgg

SEQ ID NO: 41 - DNA, 23 bp, Artificial sequence
  PAM Knock-in
cctaggcaggtggccctaggggg

SEQ ID NO: 42 - DNA, 23 bp, Artificial sequence
  PAM Knock-in
ccctaggcaggtggccctagggg

SEQ ID NO: 43 - DNA, 23 bp, Artificial sequence
  PAM Knock-in
ccctaggcaggtggccctagggg

SEQ ID NO: 44 - DNA, 23 bp, Artificial sequence
  PAM Knock-in
tccctaggcaggtggccctaggg

SEQ ID NO: 45 - DNA, 23 bp, Artificial sequence
  PAM Knock-in
cacagtgggcttggcccttctgg

SEQ ID NO: 46 - DNA, 23 bp, Artificial sequence
  PAM Knock-in
attgtgttaattattcattgagg

SEQ ID NO: 47 - DNA, 23 bp, Artificial sequence
  PAM Knock-in
taattaacacaatctcctgtggg

SEQ ID NO: 48 - DNA, 23 bp, Artificial sequence
  PAM Knock-in
aaaagttaattgggatgatatgg

SEQ ID NO: 49 - DNA, 23 bp, Artificial sequence
  PAM Knock-in
gcagccaaaagttaattgggagg

SEQ ID NO: 50 - DNA, 23 bp, Artificial sequence
  PAM Knock-in
acatggcacctgcaatcagctgg

SEQ ID NO: 51 - DNA, 23 bp, Artificial sequence
  PAM Knock-in
cctgtgtccctaccagacccagg

SEQ ID NO: 52 - DNA, 23 bp, Artificial sequence
  PAM Knock-in
acagaatggtgagaatgtaaggg

SEQ ID NO: 53 - DNA, 23 bp, Artificial sequence
  PAM Knock-in
ctattatttttgttagaggtggg

SEQ ID NO: 54 - DNA, 23 bp, Artificial sequence
  PAM Knock-in
gtgtaccttccctgaaagagggg

SEQ ID NO: 55 - DNA, 23 bp, Artificial sequence
  PAM Knock-in
tgtgtaccttccctgaaagaggg

SEQ ID NO: 56 - DNA, 23 bp, Artificial sequence
  PAM Knock-in
attttgagatagtcatagcctgg

SEQ ID NO: 57 - DNA, 23 bp, Artificial sequence
  PAM Knock-in
tcagcaagcagttttattagagg

SEQ ID NO: 58 - DNA, 23 bp, Artificial sequence
  PAM Knock-in
gcactctggaaacatacaacagg

SEQ ID NO: 59 - DNA, 23 bp, Artificial sequence
  PAM Knock-in
gagtggagtggaaggggtttggg

SEQ ID NO: 60 - DNA, 23 bp, Artificial sequence
  PAM Knock-in
tgagtggagtggaaggggtttgg

SEQ ID NO: 61 - DNA, 23 bp, Artificial sequence
  PAM Knock-in
gaattcctttccccagacccggg

SEQ ID NO: 62 - DNA, 23 bp, Artificial sequence
  PAM Knock-in
atataaagaactcaagaaggtgg

SEQ ID NO: 63 - DNA, 23 bp, Artificial sequence
  PAM Knock-in
aggtaacagggtagattagtcgg

SEQ ID NO: 64 - DNA, 23 bp, Artificial sequence
  PAM Knock-in
ctctgcatgctgaaggcttctgg

SEQ ID NO: 65 - DNA, 23 bp, Artificial sequence
  PAM Knock-in
aagctaaaacacttctgagttgg

SEQ ID NO: 66 - DNA, 23 bp, Artificial sequence
  PAM Knock-in
gatttccagagtggttgtatagg

SEQ ID NO: 67 - DNA, 23 bp, Artificial sequence
  PAM Knock-in
tacctagtgtgccggaaggtagg

SEQ ID NO: 68 - DNA, 23 bp, Artificial sequence
  PAM Knock-in
ggaccagtcgctctcagacagga

SEQ ID NO: 69 - DNA, 23 bp, Artificial sequence
  PAM Knock-in
tagaagcgcaatgtgccaaaagg

SEQ ID NO: 70 - DNA, 23 bp, Artificial sequence
  PAM Knock-in
cggatgggctgaatgggtcacgg

SEQ ID NO: 71 - DNA, 23 bp, Artificial sequence
  PAM Knock-in
gagttggatctctcctattgtgg

SEQ ID NO: 72 - DNA, 23 bp, Artificial sequence
  PAM Knock-in
gtttgaccctataaccttcacgg

SEQ ID NO: 73 - DNA, 23 bp, Artificial sequence
  PAM Knock-in
tgtagtagcctattttctcttgg

SEQ ID NO: 74 - DNA, 23 bp, Artificial sequence
  PAM Knock-in
tggcacaactggttgttatcggg

SEQ ID NO: 75 - DNA, 23 bp, Artificial sequence
  PAM Knock-in
ctggcacaactggttgttatcgg

SEQ ID NO: 76 - DNA, 23 bp, Artificial sequence
  PAM Knock-in
gtagccaacatcaaagtaaatgg

SEQ ID NO: 77 - DNA, 23 bp, Artificial sequence
  PAM Knock-in
ctgcaagggctatttctaaaagg

SEQ ID NO: 78 - DNA, 23 bp, Artificial sequence
  PAM Knock-in
gatgagcagagtttcttatatgg

SEQ ID NO: 79 - DNA, 23 bp, Artificial sequence
  PAM Knock-in
cagaggagaccaagagaacatgg

SEQ ID NO: 80 - DNA, 23 bp, Artificial sequence
  PAM Knock-in
cacttcaagacaaatggattagg

SEQ ID NO: 81 - DNA, 23 bp, Artificial sequence
  PAM Knock-in
acactggcatcagaggatacagg

SEQ ID NO: 82 - DNA, 23 bp, Artificial sequence
  PAM Knock-in
aatacccacctctgcaaaagggg

SEQ ID NO: 83 - DNA, 23 bp, Artificial sequence
  PAM Knock-in
caatacccacctctgcaaaaggg

SEQ ID NO: 84 - DNA, 23 bp, Artificial sequence
  PAM Knock-in
ctgctttctgatgggacgtaagg

SEQ ID NO: 85 - DNA, 23 bp, Artificial sequence
  PAM Knock-in
atacatgtataacttgtgcatgg

SEQ ID NO: 86 - DNA, 23 bp, Artificial sequence
  PAM Knock-in
gcacccacatagcaggtcatagg

SEQ ID NO: 87 - DNA, 23 bp, Artificial sequence
  PAM Knock-in
tcatttatgtattatgcatgtgg

SEQ ID NO: 88 - DNA, 23 bp, Artificial sequence
  PAM Knock-in
gggcgaggctgagtggttaaagg

SEQ ID NO: 89 - DNA, 23 bp, Artificial sequence
  PAM Knock-in
gtgtcctagcaaaaagggagggg

SEQ ID NO: 90 - DNA, 23 bp, Artificial sequence
  PAM Knock-in
tgtgtcctagcaaaaagggaggg

SEQ ID NO: 91 - DNA, 23 bp, Artificial sequence
  PAM Knock-in
ctctgtgctgaaggcagcagtgg

SEQ ID NO: 92 - DNA, 23 bp, Artificial sequence
  PAM Knock-in
ggagaagatggcaaaactactgg

SEQ ID NO: 93 - DNA, 23 bp, Artificial sequence
  PAM Knock-in
caagaggcatcgagattgcatgg

SEQ ID NO: 94 - DNA, 23 bp, Artificial sequence
  PAM Knock-in
ttatcctccttcatcaccaccgg

SEQ ID NO: 95 - DNA, 23 bp, Artificial sequence
  PAM Knock-in
gcggaagagggaggcagcagggg

SEQ ID NO: 96 - DNA, 23 bp, Artificial sequence
  PAM Knock-in
ggcggaagagggaggcagcaggg

SEQ ID NO: 97 - DNA, 23 bp, Artificial sequence
  PAM Knock-in
tctatgtgatgctgagctgatgg

SEQ ID NO: 98 - DNA, 23 bp, Artificial sequence
  PAM Knock-in
ctgggttctttccagatgctggg

SEQ ID NO: 99 - DNA, 23 bp, Artificial sequence
  PAM Knock-in
catatcagccttatttataatgg

SEQ ID NO: 100 - DNA, 23 bp, Artificial sequence
  PAM Knock-in
ggcctttaaggatcccaaactgg

SEQ ID NO: 101 - DNA, 23 bp, Artificial sequence
  PAM Knock-in
ggaaaaggggaggaggagagggg

SEQ ID NO: 102 - DNA, 23 bp, Artificial sequence
  PAM Knock-in
gggaaaaggggaggaggagaggg

SEQ ID NO: 103 - DNA, 23 bp, Artificial sequence
  PAM Knock-in
aatgtggaagttctgtccttggg

SEQ ID NO: 104 - DNA, 23 bp, Artificial sequence
  PAM Knock-in
taatgtggaagttctgtccttgg

SEQ ID NO: 105 - DNA, 23 bp, Artificial sequence
  PAM Knock-in
cagtgaaatttatttctgggagg

SEQ ID NO: 106 - DNA, 23 bp, Artificial sequence
  PAM Knock-in
tcacctctggcatcttaaactgg

SEQ ID NO: 107 - DNA, 23 bp, Artificial sequence
  PAM Knock-in
ctaacatggtttgacatcaaagg

SEQ ID NO: 108 - DNA, 23 bp, Artificial sequence
  PAM Knock-in
tgtttcgagacagggtttctcgg

SEQ ID NO: 109 - DNA, 23 bp, Artificial sequence
  PAM Knock-in
ttcaaggccagcctggtctacgg

SEQ ID NO: 110 - DNA, 23 bp, Artificial sequence
  PAM Knock-in
tcctagcacttgggaggcagagg

SEQ ID NO: 111 - DNA, 23 bp, Artificial sequence
  PAM Knock-in
gtaacaaggcagggccggtaggg

SEQ ID NO: 112 - DNA, 23 bp, Artificial sequence
  PAM Knock-in
ctggtaacagggcagggtgttgg

SEQ ID NO: 113 - DNA, 23 bp, Artificial sequence
  PAM Knock-in
taaatcaggaaagaaaaagaagg

SEQ ID NO: 114 - DNA, 23 bp, Artificial sequence
  PAM Knock-in
aaggcagaagaccataataatgg

SEQ ID NO: 115 - DNA, 23 bp, Artificial sequence
  PAM Knock-in
atgttatagaatgtttttagtgg

SEQ ID NO: 116 - DNA, 23 bp, Artificial sequence
  PAM Knock-in
aataagtcaccttactgtccagg

SEQ ID NO: 117 - DNA, 23 bp, Artificial sequence
  PAM Knock-in
taagaccacccaaactctgccgg

SEQ ID NO: 118 - DNA, 23 bp, Artificial sequence
  PAM Knock-in
aggaaggaaggaagggagggagg

SEQ ID NO: 119 - DNA, 23 bp, Artificial sequence
  PAM Knock-in
gacccacaaacattctaccctgg

SEQ ID NO: 120 - DNA, 23 bp, Artificial sequence
  PAM Knock-in
cttgggaggcagaggcaggcagg

SEQ ID NO: 121 - DNA, 23 bp, Artificial sequence
  PAM Knock-in
tggacttctctgaattaacctgg

SEQ ID NO: 122 - DNA, 23 bp, Artificial sequence
  PAM Knock-in
ggaagagattgtgaatggagggg

SEQ ID NO: 123 - DNA, 23 bp, Artificial sequence
  PAM Knock-in
cctatcttgggaggctgtagagg

SEQ ID NO: 124 - DNA, 23 bp, Artificial sequence
  PAM Knock-in
atctccttttgttgtcgaattgg

SEQ ID NO: 125 - DNA, 23 bp, Artificial sequence
  PAM Knock-in
caactcaccctcttaactttggg

SEQ ID NO: 126 - DNA, 23 bp, Artificial sequence
  PAM Knock-in
tagcaaaaacaaaacgaaaaggg

SEQ ID NO: 127 - DNA, 23 bp, Artificial sequence
  PAM Knock-in
atagcaaaaacaaaacgaaaagg

SEQ ID NO: 128 - DNA, 23 bp, Artificial sequence
  PAM Knock-in
gctcttcctcgtactcttttcgg

SEQ ID NO: 129 - DNA, 23 bp, Artificial sequence
  PAM Knock-in
ttgtttttgctatttccaagagg

SEQ ID NO: 130 - DNA, 23 bp, Artificial sequence
  PAM Knock-in
ttctgaataaccaaaactctcgg

SEQ ID NO: 131 - DNA, 23 bp, Artificial sequence
  PAM Knock-in
tacacacccaggtgtcccagggg

SEQ ID NO: 132 - DNA, 23 bp, Artificial sequence
  PAM Knock-in
ggcatttcatctaaggttcttgg

SEQ ID NO: 133 - DNA, 23 bp, Artificial sequence
  PAM Knock-in
atgcatatttgcttatgtaaagg

SEQ ID NO: 134 - DNA, 23 bp, Artificial sequence
  PAM Knock-in
atgaccaacaaagggtggtaagg

SEQ ID NO: 135 - DNA, 23 bp, Artificial sequence
  PAM Knock-in
aagaagatacaaatttatagcgg

SEQ ID NO: 136 - DNA, 23 bp, Artificial sequence
  PAM Knock-in
agaagatacaaatttatagcagg

SEQ ID NO: 137 - DNA, 23 bp, Artificial sequence
  PAM Knock-in
tttttggtatatattttaggagg

SEQ ID NO: 138 - DNA, 23 bp, Artificial sequence
  PAM Knock-in
tttttttcctcctgcaaatctgg

SEQ ID NO: 139 - DNA, 23 bp, Artificial sequence
  PAM Knock-in
aggaaccgccctgaggagagcgg

SEQ ID NO: 140 - DNA, 23 bp, Artificial sequence
  PAM Knock-in
ctgccacatgggggcaacagagg

SEQ ID NO: 141 - DNA, 23 bp, Artificial sequence
  PAM Knock-in
gaaggatactgacgtccaggcgg

SEQ ID NO: 142 - DNA, 23 bp, Artificial sequence
  PAM Knock-in
tcctgctaaaaaattactaacgg

SEQ ID NO: 143 - DNA, 23 bp, Artificial sequence
  PAM Knock-in
ggctccacaccccgtggtcgagg

SEQ ID NO: 144 - DNA, 23 bp, Artificial sequence
  PAM Knock-in
ttctgtttacatgggctgtgtgg

SEQ ID NO: 145 - DNA, 23 bp, Artificial sequence
  PAM Knock-in
attgctggtcaacaatggattgg

SEQ ID NO: 146 - DNA, 23 bp, Artificial sequence
  PAM Knock-in
tagacttgagcaatgacagaggg

SEQ ID NO: 147 - DNA, 23 bp, Artificial sequence
  PAM Knock-in
atagacttgagcaatgacagagg

SEQ ID NO: 148 - DNA, 23 bp, Artificial sequence
  PAM Knock-in
gggtcctctggaagaacagcagg

SEQ ID NO: 149 - DNA, 23 bp, Artificial sequence
  PAM Knock-in
cacgttgatcaatggaattaagg

SEQ ID NO: 150 - DNA, 23 bp, Artificial sequence
  PAM Knock-in
ccaccatttccaatttcaagtgg

SEQ ID NO: 151 - DNA, 23 bp, Artificial sequence
  PAM Knock-in
gaaacaatcctgaacagtgaagg

SEQ ID NO: 152 - DNA, 23 bp, Artificial sequence
  PAM Knock-in
gaattttggtgggtatctcacgg

SEQ ID NO: 153 - DNA, 23 bp, Artificial sequence
  PAM Knock-in
cctgtctgtctctgcctctctgg

SEQ ID NO: 154 - DNA, 23 bp, Artificial sequence
  PAM Knock-in
cagagagagacagagacagaggg

SEQ ID NO: 155 - DNA, 23 bp, Artificial sequence
  PAM Knock-in
gcagagagagacagagacagagg

SEQ ID NO: 156 - DNA, 23 bp, Artificial sequence
  PAM Knock-in
cacaccacaacccctacacctgg

SEQ ID NO: 157 - DNA, 23 bp, Artificial sequence
  PAM Knock-in
tatgtaaaaatgacacgattagg

SEQ ID NO: 158 - DNA, 23 bp, Artificial sequence
  PAM Knock-in
gggatgttgtgtaggagcttggg

SEQ ID NO: 159 - DNA, 23 bp, Artificial sequence
  PAM Knock-in
aatgggtctctagttgcgggtgg

SEQ ID NO: 160 - DNA, 23 bp, Artificial sequence
  PAM Knock-in
tggttacagtagccgcagcacgg

SEQ ID NO: 161 - DNA, 23 bp, Artificial sequence
  PAM Knock-in
ccgctttacttctgatagattgg

SEQ ID NO: 162 - DNA, 23 bp, Artificial sequence
  PAM Knock-in
aacctaagacaggtacagtccgg

SEQ ID NO: 163 - DNA, 23 bp, Artificial sequence
  PAM Knock-in
gtctggaagcttttctgtgtggg

SEQ ID NO: 164 - DNA, 23 bp, Artificial sequence
  PAM Knock-in
tattccctgatagatcataaggg

SEQ ID NO: 165 - DNA, 23 bp, Artificial sequence
  PAM Knock-in
gtattccctgatagatcataagg

SEQ ID NO: 166 - DNA, 23 bp, Artificial sequence
  PAM Knock-in
agatagaaggagatggtacatgg

SEQ ID NO: 167 - DNA, 23 bp, Artificial sequence
  PAM Knock-in
gatttttctctatagtgcagcgg

SEQ ID NO: 168 - DNA, 23 bp, Artificial sequence
  PAM Knock-in
tatcccttgagcagtaaaggagg

SEQ ID NO: 169 - DNA, 23 bp, Artificial sequence
  PAM Knock-in
ttttctggtagaattttatgagg

SEQ ID NO: 170 - DNA, 23 bp, Artificial sequence
  PAM Knock-in
tttaaaaccctgtttccagcagg

SEQ ID NO: 171 - DNA, 23 bp, Artificial sequence
  PAM Knock-in
ttccacaaaatagaaacaaaagg

SEQ ID NO: 172 - DNA, 23 bp, Artificial sequence
  PAM Knock-in
gcattaatcccttctaagggcgg

SEQ ID NO: 173 - DNA, 23 bp, Artificial sequence
  PAM Knock-in
ggagttccttgagccactcaagg

SEQ ID NO: 174 - DNA, 23 bp, Artificial sequence
  PAM Knock-in
actcaagtctaaaaggatcaagg

SEQ ID NO: 175 - DNA, 23 bp, Artificial sequence
  PAM Knock-in
acttatagaggagaaagtggggg

SEQ ID NO: 176 - DNA, 23 bp, Artificial sequence
  PAM Knock-in
ttcagggaacctggtgagggagg

SEQ ID NO: 177 - DNA, 23 bp, Artificial sequence
  PAM Knock-in
acaggacaatgtgggtagaaggg

SEQ ID NO: 178 - DNA, 23 bp, Artificial sequence
  PAM Knock-in
aacaggacaatgtgggtagaagg

SEQ ID NO: 179 - DNA, 23 bp, Artificial sequence
  PAM Knock-in
ttttagcagaagagcattttggg

SEQ ID NO: 180 - DNA, 23 bp, Artificial sequence
  PAM Knock-in
agagaaatcagatctcagcgagg

SEQ ID NO: 181 - DNA, 23 bp, Artificial sequence
  PAM Knock-in
cttaataaccgacatcattatgg